SEQUENCE LISTING (2 sequences)

SEQ ID NO: 1 - DNA, 431 bp, Penicillium brevicompactum
atggggggttctcatcatcatcatcatcatggtatggctagcatgactggtggacagcaaatgggtcgggatctgtacgacgatgacgataaggatccaacccttatgtctaccgctgagctcgctgtctcttacgccgccctcatcctggccgatgacggtattgaggtctccgccgacaagatccagaccatcctcggtgccgccaaggtccaggaggttgagcccatctgggccaccatcttcgccaaggccctcgagggtaaggacatcaaggagatcctgaccaacgtcggctccgctggtcccgccaccgccggtgcccccgccgctgctggtgccgccgctcccgctgaggagaagaaggaggagaaggaagaggagaaggaggagtccgatgaggacatgggcttcggtctcttcgactaagc

SEQ ID NO: 2 - protein, 107 aa, Penicillium brevicompactum
MSTAELAVSYAALILADDGIEVSADKIQTILGAAKVQEVEPIWATIFAKALEGKDIKEILTNVGSAGPATAGAPAAAGAAAPAEEKKEEKEEEKEESDEDMGFGLFD

(19) United States
(12) Patent Application Publication
(10) Pub. No.: US 2007/0077255 A1
(43) Pub. Date: Apr. 5, 2007

(54) Isolation and characterization of a cDNA clone encoding one IgE-binding fragment of Penicillium brevicompactum

(76) Inventors: M. Serdal Sevinc, Ottawa (CA); Hari M. Vijay, Ottawa (CA)

Correspondence Address: ADE & COMPANY INC., 2157 Henderson Highway, WINNIPEG, MB, R2G1P9, CA

(21) Appl. No.: 11/528,679

(22) Filed: Sep. 28, 2006

Related U.S. Application Data
(60) Provisional application No. 60/723,455, filed on Oct. 5, 2005.

(51) Int. Cl. A61K 39/00; C07H 21/04; C07K 14/385

(52) U.S. Cl. 424/185.1; 530/350; 536/23.7

(57) ABSTRACT

Pen b 26 was isolated as an allergen of Penicillium brevicompactum, which is known to be one of the major source of indoor fungal allergies. Pen b 26 was isolated and cloned into Escherichia coli as an N-terminus his-tagged fusion protein, which had a calculated molecular weight of 14.9 kDa while the native Pen b 26 was 11 kDa. The over-expressed fusion protein migrated at about 20 kDa on SDS-PAGE although its analysis by mass spectroscopy confirmed its calculated size. The IgE antibodies in the sera of >25% Penicillium-allergic individuals showed positive reaction to the purified fusion protein. Pen b 26 was also identified as a 60S ribosomal P1 phospho-protein. This invention includes a cDNA sequence from which the recombinant allergen was produced.

### PRIOR APPLICATION INFORMATION

This application claims the benefit of U.S. Provisional Application No. 60/723,455, filed Oct. 5, 2005.

### BACKGROUND OF THE INVENTION

1. Field of the Invention

This invention relates to an allergen of Penicillium brevicompactum, and specifically to a cDNA sequence encoding an allergenic protein of P. brevicompactum.

2. Discussion of the Prior Art

Airborne allergic diseases are estimated to affect about 10-20% of North American population. Major sources of airborne allergies are pollens of various plants, spores of fungi and debris of insects and domestic animals. Molecular characterization of allergens is of significant value to the development of more efficacious and specific diagnostic and therapeutic techniques. Allergic reactions are induced by the production of IgE class antibodies to the allergens because of the inability of the body to produce neutralizing IgG class antibodies. Allergenic reactions to aeroallergens include conjunctivitis, rhinitis and asthma.

The accepted treatment for allergies at present involves hyposensitization of patients by inoculating with the extracts of specific allergens to which the patient is allergic. Theoretically, hyposensitization process is thought to provoke production of IgG class antibodies to neutralize the allergens by competing for allergen with effector cell-bound IgE as well as by suppressor T-cell induction. However, most of the time the extracts used in the hyposensitization process are of lower value because they contain impurities.

P. brevicompactum has been reported to be one of the major indoor aero-allergens (Shen H. D. et al. Int Arch Allergy Immunol. 1999;119(4):259-264). Molecular characterization of these allergenic proteins from this mold is essential for a better understanding of the pathogenesis of atopic diseases and also for developing immunoassays to quantify indoor and outdoor mold and other aero-allergens. Allergens purified using conventional procedures are often obtained in low yields and tend to be of inconsistent quality. In particular, fungal allergens are subject to considerable variation between isolates of the same species, due to the fact that molds rapidly adapt to new environments. Stable and consistently pure allergenic proteins can be prepared using recombinant DNA techniques. Expression of these proteins in expression systems, such as Pichia pastoris or Escherchia coli would yield allergens in large quantities. In our laboratory, major and minor allergens of Alternaria alternata and Cladosporium herbarum have been cloned (De Vouge M. W. et al. Int. Arch. Allergy Immunol 1996;111 (4):385-395; Zhang L, et al. J Immunol 1995;154 (2);710-717). Hence, in the present study, a similar strategy was used to clone and express allergenic components of P. brevicompactum. A phage cDNA library of P. brevicompactum was screened using human atopic serum IgE. The clones were expressed and their protein products were characterized by immunological techniques. One of the isolated clones designated as Pen b 26 according to the recommendations of the International Union of Immunological Societies (King T. P. et al. Allergy 1995;50(9):765-774) will be studied in this paper.

Allergens are prepared most commonly using crude extracts (conventional techniques) or gene cloning techniques if the amino acid sequence is known.

Allergens purified using conventional procedures are often obtained in low yields and tend to be of inconsistent quality. Impurities in these extracts may cause a number of adverse effects, including anaphylactic shock. Particularly, fungal allergens are subject to considerable variation between isolates of the same species, due to the fact that molds rapidly adapt to new environments. Gene cloning techniques are superior to produce unlimited quantities of the products of the highest purity. However, the gene has to be cloned and amino acid sequence of the protein has to be determined. Furthermore, the cloned gene has to be expressed appropriately, i.e. allergic epitopes have to be preserved in the recombinant protein.

### GENERAL DESCRIPTION OF THE INVENTION

In the western world, the majority of people spend more than 90% of their life in indoor environments. These air-controlled environments often allow growth of indoor mold species in the presence of excess humidity. Excessive growth of indoor mold species creates numerous health problems in sensitive individuals, including rhinitis, conjunctivitis, pulmonary bleeding and extrinsic bronchial asthma. Major indoor mold allergens include species of Penicillium, Altemaria, Cladosporium and Aspergillus (Kurup V. P. 2003, Curr Allergy Asthma Rep 3: 416423; Kurup V. P. et al. 2002, Int Arch Allergy Immunol 129: 181-188). Penicillium is one of the most frequently encountered and diverse mold genera found in indoor air. The abundance of allergenic Penicillium species also correlates with the increased incidence of childhood asthma and mold allergies. In our laboratory, 14 Penicillium species were investigated for their allergenic components (Vijay H. M. et al. 2002, The 7^{th} Int Congr Aerobiol, Montebello). Among them, P. brevicompactum, P. viridicatum, P. janthinellum, P. citrinum and P. oxalicum were found to be highly allergenic. P. brevicompactum has been reported to be one of the major indoor aero-allergens (Shen H. D. et al. 1999, Int Arch Allergy Immunol 119: 259-264).

Molecular characterization of these allergenic proteins is essential for a better understanding of the pathogenesis of atopic diseases and also for developing immunoassays to quantify indoor and outdoor mold and other aero-allergens. Allergens purified using conventional procedures are often obtained in low yields and tend to be of inconsistent quality. In particular, fungal allergens are subject to considerable variation between isolates of the same species, due to the fact that molds rapidly adapt to new environments. Stable and consistently pure allergenic proteins can be prepared using recombinant DNA techniques. Expression of these proteins in expression systems, such as Pichia pastoris would yield allergens in large quantities. In our laboratory, major and minor allergens of Altemaria alternata and Cladosporium herbarum have been cloned (De Vouge M. W. et al. 1996, Int Arch Allergy Immunol 111: 385-395; Zhang L. et al. 1995, J Immunol 154: 710-717). Hence, in the present study, a similar strategy was used to clone and express allergenic components of P. brevicompactum. A phage cDNA library of P. brevicompactum was screened using human atopic serum IgE. The clones were expressed and their protein products were characterized by immunological techniques. One of the isolated clones designated as Pen b 26 according to the recommendations of the International Union of Immunological Societies (King T. P. et al. 1995, Allergy 50: 765-774).

### SUMMARY OF THE INVENTION

There is disclosed herein a nucleotide sequence encoding the cDNA sequence of Pen b 26 allergen (also known as 60S ribosomal P1 phospho-protein) of Penicillium brevicompactum.

There is disclosed herein a polypeptide sequence corresponding to the open reading frame of Pen b 26 cDNA of Penicillium brevicompactum, from bases 38 to 358 (amino acids 1-107)

In a first aspect of the invention, there is provided an isolated nucleic acid sequence comprising nucleotides 1-455 of SEQ ID NO: 1.

In a second aspect of the invention, there is provided an isolated peptide comprising 6 or more consecutive amino acids of SEQ ID NO: 2.

### BRIEF DESCRIPTION OF THE DRAWINGS

This invention will be described in further details with reference to the accompanying drawings, wherein:

FIG. 1. shows the purified clone Pen b 26 from a bacteriophage cDNA library of P. brevicompactum. a) Immuno-plaque hybridization and b) Western blot analysis of the purified phage extracts of the allergenic clone Pen b 26. Normal (N) and atopic (A) sera.

FIG. 2. shows the transcript of the Pen b 26 clone (SEQ ID NO: 1) and the predicted amino acid sequence (SEQ ID NO: 2) (italicised). Denotations: start (ATG), stop (TAA) codons and polyadenylation signals (AAA AAA) are underlined. Potentially phosphorylated residues T-29 and S-97 are in bold.

FIG. 3. shows the analysis of clone Pen b 26 by a) PCR using flanking M13 forward and reverse primers and b) Northern blot hybridization probed with Pen b 26 cDNA.

FIG. 4. shows the graphical output of the secondary structure analysis of Pen b 26 protein of P. brevicompactum using PSIPRED software.

FIG. 5. shows the hydrophobicity and hydrophilicity plots of Pen b 26 protein of P. brevicompactum.

FIG. 6. shows the amino acid sequence homology among 60S ribosomal proteins from different fungal sources and Pen b 26 protein of Penicillium brevicompactum. Sequences: 1-Penicillium brevicompactum Pen b 26 (GeneBank Accession No. AY786077), 2-Aspergillus fumigatus (AF268869.1), 3-Neurospora crassa (XM_{—}331351.1), 4-Cladosporium herbarum (Cla h 12) (AAMCM01000433.1), 5-Alternaria alternata (Alt a 12) (X84216.1), 6-Schizosaccharomyces pombe (P1-alpha 1) (NC_{—}003423.1). Consensus symbols: ! is anyone of I, V; # is anyone of N, D, Q, E, B, Z.

FIG. 7. shows the time-course expression of clone pTrcHisTOPO-Pen b 26 in Escherichia coli after induction with IPTG. Lanes: 1-MW markers; 2-0 min, 3-30 min, 4-1 h, 5-2 h, 6-3 h, 7-4 h, 8-5 h, 9-6 h, 10- Purified Pen b 26 (2 μg/lane).

FIG. 8. shows the mass spectral analysis of Pen b 26 by electrospray and MALDI-TOF.

FIG. 9. shows the immunoblot analysis of Pen b 26 using Human antibody against ribosomal P antigen.

FIG. 10. shows the prevalence of allergies against Pen b 26 among Penicillium-allergic patients. Positive lanes: Panel A: 2, 6, 7; Panel B: 3, 4: Panel C: 2, 8. Total: 7 positives, 7/27×100=26% positive.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

The abundance of allergenic Penicillium species has been associated with an increased incidence of childhood asthma and pulmonary bleeding. Penicillium brevicompactum has been identified as the most prevalent indoor species of this genus. However, detailed studies on the allergens of the ubiquitous Penicillium species are still lacking. For the characterization of allergens of prevalent Penicillium species, molecular cloning of the allergen genes of P. brevicompactum was performed.

Penicillium species have long been associated with indoor mold allergies. A number of allergens have been isolated from various Penicillium species. These include Pen c 1 (Su N.Y. et al. 1999, Eur J Biochem 261: 115-123, 821), Pen c 2 (Chow L. P. et al. 1999, Biochem J 341: 51-59) and Pen c 3 (Shen H. D. et al. 2000, J Allergy Clin Immunol 105: 827-833) of P. citrinum, Pen o 18 of P. oxalicum (Shen H. D. et al. 2001, J Lab Clin Med 137: 115-124), Pen ch 13 (a.k.a. Pen n 13) (Chow L. P. et al. 2000, Biochem Biophys Res Commun 269: 14-20; Chou H. et al. 2002, Int Arch Allergy Immunol 127: 15-26), Pen ch 18 (Shen H. D. et al. 2003, Allergy 58: 993-1002) of P. chrysogenum. However, not all Penicillium species are well-studied and a number of allergens from Penicillium species are awaiting discovery. In this study, we attempted to isolate and clone the allergenic proteins from P. brevicompactum. Isolation of cloned allergens or allergenic clones by the use of atopic sera has been a method of choice. In this study, a phage cDNA library of P. brevicompactum has been screened with sera obtained from individuals allergic to this mold. A clone designated as Pen b 26 was successfully isolated using atopic sera.

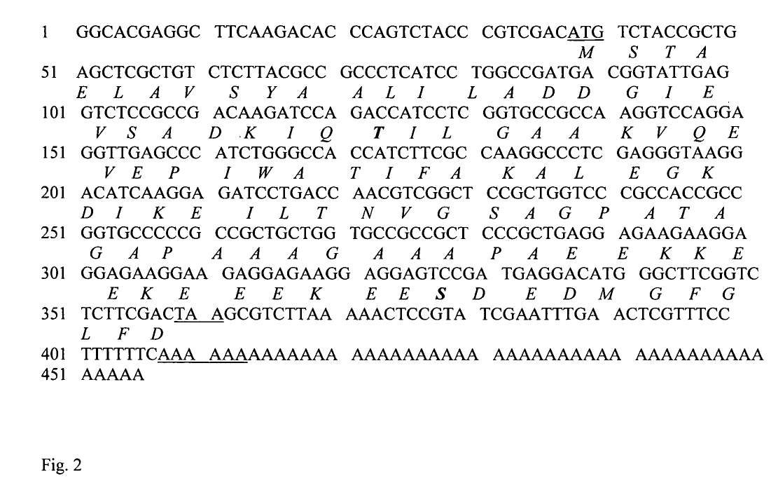

In one embodiment, there is provided a nucleic acid molecule encoding Pen b 26 protein corresponding to nucleotides 1-455 of FIG. 2, (SEQ ID NO. 1). In another embodiment, there is provided a purified or isolated Pen b 26 peptide having an amino acid sequence as shown in FIG. 2 (amino acids 1-107 of FIG. 2, SEQ ID NO. 2). In yet other embodiments, there is provided a nucleic acid molecule deduced from the amino acid sequence of the Pen b 26 peptide.

The Pen b 26 protein appeared to have a low molecular weight of only 11 kDa which was confirmed by Western blotting, DNA sequence analysis, PCR and Northern blot hybridization. BLAST analysis of its amino acid sequence indicated that this protein had strong sequence homology to a well-known 60S ribosomal P1 protein from several species of fungi and also several other eukaryotic organisms.

Ribosomal P proteins are phospho-proteins and characterized from a variety of species (Matheson A. T. et al. 1980, in Ribosomes: Structure, Function and Genetics, Chambliss et al., eds., pp 297-332, University Park Press: Baltimore; Tchorzewski M. 2002, Int J Biochem Cell Biol 34: 911-915; Rich B. E. and Steitz J. A. 1987, Mol Cell Biol 7: 4065-4074; Remacha M. et al. 1995, Mol Cell Biol 15: 47544762; Santos C. and Ballesta J. P. 1994, J Biol Chem 269: 15689-15696; Bailey-Serres J. et al. 1997, Plant Physiol 114: 1293-1305; Tchorzewski M. et al. 2003, Biochemistry 42: 3399-3408). In humans, P1 protein, together with two other acidic P proteins, namely P2 and P0, form a distinct lateral protuberance on the 60S ribosomal subunit (Rich B. E. and Steitz J. A. 1987). They interact with elongation factors, Ef1 and Ef2, and their level of phosphorylation is a regulatory mechanism for the overall rate of translation (Chen A. et al. 2002, Nat Med 8: 1421-1426). In the yeast Saccharomyces cerevisiae, the P proteins, unlike most riboproteins, are found in multi copies and form a pentameric complex with two isomeric copies of P1 (α/β) and P2 (α/β) joined with one copy of P0 (Tchorzewski M. et al. 2003). Yeast strains with all four disrupted P1 and P2 genes are viable, indicating that these genes are not absolutely essential, although these cells exhibit slower growth rate, cold-sensitivity and inability to sporulate (Remacha M. et al. 1995). In contrast, P0 protein is absolutely essential for cell viability and ribosome activity (Santos C. and Ballestra J. P. 1994). Actively growing yeast cells synthesize higher numbers of P proteins than the stationary cells (Saenz-Robles M. T. et al. 1990, Biochim Biophys Acta 1050: 51-55). Although most proteins in the 60S ribosome are basic, P proteins are among the few acidic proteins. P1 and P2 proteins, after synthesis, shuttle between cytoplasmic pool and the ribosome whereas the P0 protein remains exclusively in the ribosomes (Santos C. and Ballestra J. P. 1994, J Biol Chem 269: 15689-15696).

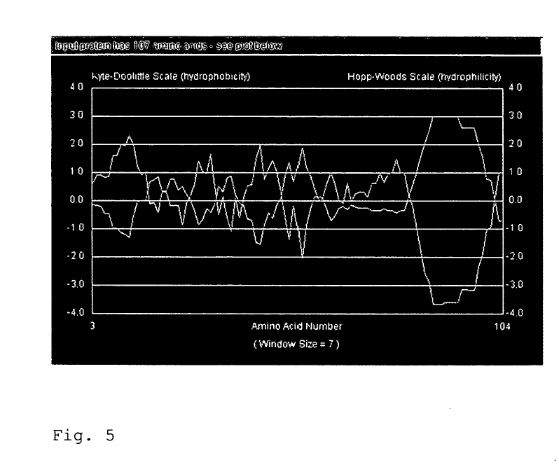

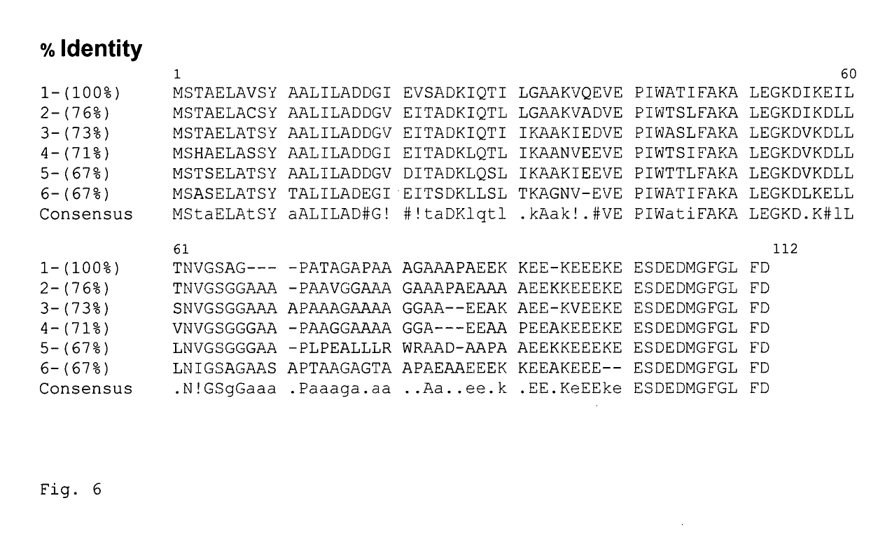

The presence of autoantibodies against the ribosomal P proteins in autoimmune diseases (Fabien N. et al. 1999, J Autoimmun 13: 103-110) and their association in IgE-mediated allergic diseases (Appenzeller U. et al. 1999, Int Arch Allergy Immunol 118: 193-196) confirm the allergenicity of Pen b 26 protein of P. brevicompactum. All P proteins seem to contain an important epitope structure in their C-terminus that is recognized by the antibodies in the sera of individuals with autoimmune diseases, such as systemic lupus erythematosus (Bonfa E. et al. 1987, N Engl J Med 317: 265-271), protozoan infections like leishmaniosis (Soto M. et al. 1993, J Biol Chem 268: 21835-21843), and Chagas' heart disease (Mesri E. A. et al. 1990, J Clin Microbiol 28: 1219-1224). In addition to the common C-terminus epitope, P proteins contain epitope regions in their N-terminal regions (Fabien N. et al. 1999). Based on hydrophilicity plots (FIG. 5) and comparison of the sequence homologies from different 60S ribosomal P1 proteins (FIG. 6), the C-terminus of Pen b 26 also contains this common epitope region. The cellular location of P proteins has recently been re-investigated and in addition to cytoplasmic location, fully phosphorylated P proteins were also found on the cell wall (Boguszewska A. et al. 2002, Biol. Cell 94: 139-146). The new data may explain their allergenicity. Since P1 proteins are on the fungal cell wall, they can easily induce allergies by direct contact to the skin or nasal membranes of the sensitive individuals without lysis of the cell.

The secondary structure analysis of Pen b 26 protein conforms to the secondary structure of the other P proteins. It has high alpha-helical content (>50%) in the N-terminus and a flexible hinge region. The central region and its high alanine content (˜20%) and acidic nature (high in acidic residues and low pl), in conjunction with the predicted phosphorylation site in the conserved C-terminal epitope region, like other known P1 proteins support the hypothesis that the isolated clone Pen b 26 is indeed an acidic ribosomal P1 protein. This clone does react with atopic sera from allergenic individuals and has structure similar to other acidic ribosomal P1 proteins.

It is of note that it is well known in the art that some modifications and changes can be made in the structure of a polypeptide without substantially altering the biological function of that peptide, to obtain a biologically equivalent polypeptide. In one aspect of the invention, the above-described peptides may include peptides that differ by conservative amino acid substitutions. The peptides of the present invention also extend to biologically equivalent peptides that differ by conservative amino acid substitutions. As used herein, the term “conserved amino acid substitutions” refers to the substitution of one amino acid for another at a given location in the peptide, where the substitution can be made without substantial loss of the relevant function, in this case, the folding of the epitope. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. It is important to note that in many embodiments of the invention, the ‘function’ of the peptides relates to their immunogenicity. That is, as discussed below, the purified or isolated Pen b 26 are used to generate antibodies for example for detecting the presence of Penicillium and for hyposensitizing allergic individuals. As such, substitutions that are outside of the immunogenic regions of the Pen b 26 peptide, that is, in epitope-free regions, are more likely to be tolerated without a loss of ‘function’, as will be apparent to one of skill in the art.

In some embodiments, conserved amino acid substitutions may be made where an amino acid residue is substituted for another having a similar hydrophilicity value (e.g., within a value of plus or minus 2.0), where the following may be an amino acid having a hydropathic index of about −1.6 such as Tyr (−1.3) or Pro (−1.6)s are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2) Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Len (+3.8); Phe (+2.8); Cys (+2.5); Met (+1.9); Ala (+1.8); Gly (−0.4); Thr (−0.7); Set (−0.8); Trp (−0.9); Tyr (−1.3); Pro (−1.6); His (−3.2); Glu (−3.5); Gln (−3.5); Asp (−3.5); Am (−3.5); Lys (−3.9); and Arg (−4.5).

In alternative embodiments, conserved amino acid substitutions may be made where an amino acid residue is substituted for another in the same class, where the amino acids are divided into non-polar, acidic, basic and neutral classes, as follows: non-polar: Ala, Val, Len, Ile, Phe, Trp, Pro, Met; acidic: Asp, Glu; basic: Lys, Arg, His; neutral: Gly, Ser, Thr, Cys, Asn, Gln, Tyr.

In some embodiments, there is provided an expression system comprising a nucleic acid molecule as described above encoding Pen b 26 or a variant thereof as described above operably linked to control sequences suitable for directing expression of the nucleic acid in a suitable host cell. Examples of such expression systems are well-known in the art. As will also be known by one of skill in the art, peptides so expressed can be easily isolated and/or purified using a variety of means, for example, size exclusion chromatography, charge-based chromatography or antibody columns, to name a few. Alternatively, the peptide of interest may be fused to a tag or ‘affinity handle’, that is, an antigenic eptiope used for the purification of peptides, such as the histidine tag described herein (SEQ ID NO: 3 and SEQ ID NO: 4).

In other embodiments, an immunogenic fragment of the Pen b 26 peptide or a variant thereof is purified or isolated as discussed above. As will be well-known to one of skill in the art, software programs for the prediction of epitopes are well known in the art. It is further noted that as discussed below, the C terminal region of Pen b 26 is highly immunogenic. Thus, in one embodiment of the invention there is provided a peptide comprising 6 or more, 7 or more, 8 or more, 9 or more or 10 or more consecutive amino acids of SEQ ID NO: 2 for use as an immunogenic fragment of Pen b 26, which are discussed in greater detail herein.

In some embodiments, such epitopes may be chemically fused to suitable carrier proteins known in the art or may be genetically fused to suitable carrier proteins known in the art.

In other embodiments, Pen b 26 peptide or a variant thereof or an immunogenic fragment thereof may be used to inoculate a suitable animal for the purpose of generating polyclonal or monoclonal antibodies against Pen b 26. These antibodies may be used on a variety of platforms known in the art for the detection of Penicillium. In these embodiments, antibodies may be mounted onto a support or platform which is arranged for aerosol detection of allergens so that Penicillium levels, for example, Penicillium brevicompactum levels, in a given area can be determined.

In another embodiment of the invention, purified and/or isolated Pen b 26 or a variant thereof or an immunogenic fragment thereof is used to hyposensitize an individual in need of such treatment to exposure to Penicillium by inoculating or injecting said individual with an effective amount of a purified Pen b 26. As will be appreciated by one of skill in the art, an ‘effective amount’ is an amount sufficient to provoke production of IgG antibodies against Pen b 26 in the individual. An individual in need of such treatment is an individual who is either allergic to or at risk of developing an allergy to Penicillium, for example, P. brevicompactum.

The invention will now be further illustrated by way of examples. However, the examples are for illustrative purposes and are not necessarily limiting.

Materials and Methods

Molecular biological techniques were performed according to the standard protocols unless otherwise stated (Sambrook J. and Russel D. W. 2001, Molecular Cloning: A Laboratory Manual (3^{rd} Edition), Cold Spring Harbor Laboratory Press: Cold Spring Harbor, N.Y.; Ausubel F.M. et al. 1990, Current Protocols in Molecular Biology, Greene Publishing/Wiley-Interscience: New York).

RNA Isolation and Construclion of the cDNA Library of P. brevicompactum

An isolate of P. brevicompactum was grown in liquid malt extract broth (Difco, Detroit, Mich., USA) at 22° C. Mycelia were harvested on days 3, 5, 7 and 12 by washing (4×) with 0.6 M MgSO₄ and stored at −85° C. For RNA extraction, the mycelial mass was ground using a mortar and pestle under liquid nitrogen. Total RNA was extracted from the mycelial powder with TRIZOL® Reagent (Invitrogen Life Technologies, Carlsbad, Calif., USA) according to the manufacturer's instructions. Briefly, the mycelial powder was resuspended in TRIZOL Reagent with the addition of 10 vol of this reagent. The mixture was homogenized using a Polytron® PT 10-35 (Kinematica, Littau/Lucerne, Switzerland) 8× with short pulses and intervals. Chloroform was added to 10% of the total volume. The mixture was centrifuged at 12,000 g for 10 min. The supernatant was collected and RNA was precipitated with the addition of equal volumes of isopropanol and high salt solution (0.8 M sodium citrate and 1.2 M sodium chloride) to 50% of the final volume. The RNA pellets were washed once with 80% ethanol, dried briefly and resuspended in RNAse-free water. The cDNA library of P. brevicompactum was constructed in Uni-ZAP® XR vector using the total RNA and oligo(dT) linkers by Stratagene (La Jolla, Calif., USA). Amplification and screening of the cDNA were performed in Escherichia coli XL1-blue host strain.

Antisera and Immunoscreening of P. brevicompactum CDNA Library

Immuno plaque hybridization assay was carried out using STRATAGENE picoBlue™ immunoscreening kit. Sera from individuals recognized as P. brevicompactum sensitive by positive skin test were collected and assayed by a radioallergosorbent test (RAST) using P. brevicompactum cultures prepared in our laboratory (Vijay H. M. et al. 2002. Allergenic and mutagenic characterization of 14 Penicillium species. The 7^{th} Int. Cong. Aerobiol. Montebello). Sera scoring 20% or greater by RAST were utilized in pools for immunoscreening and individually when assessing reactivity of recombinant fusion proteins. For the initial high density screening, the library was plated at 50,000 plaque-forming units per 150-mm Petri plate. Plaques were lifted by placing duplicate isopropyl thio-β-D-galactoside (IPTG) absorbed nitrocellulose membranes (150 mm diameter) onto the growing plaques after 2 and 5 h, respectively. The membranes were first blocked in 1% BSA (bovine serum albumin) in TBST (50 mM Tris-HCl pH 7.5, 150 mM NaCl, 0.05% Tween-20) for 1 h at RT, followed by incubation overnight at 4° C. in an atopic serum pool (pooled from three sensitive individuals). Normal human serum was used as negative control. To eliminate background nonspecific cross-reactivity, both atopic and control sera were pre-absorbed with Escherichia coli lysate, diluted 5-fold in the above blocking solution. The membranes were washed 4× for 5 min each with TBST, and incubated with 1/500 dilution of a mouse monoclonal anti-human IgE-alkaline phosphatase conjugate (BD Biosciences, Franklin Lakes. N.J., USA). The membranes were washed as described earlier and incubated with 0.3 mg/ml NBT (nitroblue tetrazolium) and 0.15 mg/ml BClP:(5-bromo-4-chloro-3-indolyl phosphate) in color development buffer (100 mM Tris-HCl, pH 9:5, 100 mM NaCl, 5 mM MgCl₂) until positive plaques were clearly: visible (5-10 min). Further color development was stopped by washing the membranes with distilled water once and incubating in 20 mM Tris-HCl, pH 2.9 and 1 mM EDTA. Positive clones were identified by comparing the orientation of the plaques on the primary- and secondary-lifted membranes. The positive clones were further purified by serial dilution until all the plaques showed positive reaction in the same plate.

Allergen Expression in Purified Phage Clone

The IgE-positive phage clone was inoculated with 100 ml of mid-log growing E. coli XL1-Blue host cells in a 500-ml flask. After 30 min incubation, IPTG was added to induce the expression of cloned protein. The culture was centrifuged and the supernatant containing the phage was collected. The phages were pelleted by the addition of 20% PEG-1.5 M NaCl solution to 30% of the original culture volume, followed by centrifugation. The resulting pellet was briefly sonicated in a microcentrifuge tube with a micro-tip probe at medium intensity (3×for 10 s). About 10 μg sonicated sample in loading buffer containing β-mercaptoethanol was heated to 95° C. for 7 min. The sample was electrophoresed in 12% polyacrylamide using a vertical slab mini gel SDS-PAGE (Laemmli U.K. 1970, Nature 227: 680-685) system (BIO-Rad, Mississauga, Ont., Canada). Broad range molecular weight markers (BIO-Rad) were used for size determination of protein bands. The gel was electro-transferred onto PVDF membrane (Matsudaira P. 1987, J Biol Chem 262: 10035-10039). Subsequently, standard Western blot hybridization was performed using primary atopic antibodies and secondary monoclonal anti IgE-AP antibodies. In a separate but identical gel, proteins were staining with Coomassie Brilliant Blue R-250 to ensure that sufficient amount of protein was present.

Isolation of cDNA Clones

The excision of the cloned fragments from the phage vector was performed according to the manufacturer's manual (Stratagene). Briefly, E. coli XL1-Blue cells were co-infected with the phage clones and helper phage ExAssist for 15 min. The co-infected host cells were heated to 70° C. for 20 min to kill the host cells and release the phagemids. The heated cell-phage mixture was centrifuged, and the supernatant containing phagemids was incubated with E coli SOLR cells to produce plasmid clones.

DNA Sequencing

For DNA sequencing, plasmid DNA was purified using Qiagen miniprep kit. Asymmetric polymerase chain reaction (PCR) was carried out using fiuorescein-labelled ddNTPs and M13 forward and reverse primers with Big Dye Terminator version 3.1 Cycle sequencing kit (Applied Biosystems, Foster City, Calif., USA) and ABI 310/3100 Genetic Analyser instrumentation. The results were analysed using the software of Sequencher (Gene Codes, Ann Arbor, Mich., USA). The open reading frame was detected using GENSCAN software (Burge C. and Karlin S. 1997, J Mol Biol 268: 78-94).

Amplification of cDNA Clones by PCR

The cDNA sequence from the pBluescript clones were amplified using flanking M13 forward and reverse primers with hot-start Taq polymerase (Invitrogen). PCR was performed by initial denaturation at 94° C. for 3 min, followed by 30 cycles of 30 s denaturation at 94° C., 15 s annealing at 55° C. and 30 s extension at 72° C. A 7 min extension in the end of the 30 cycles was also incorporated. For the expression of the cloning allergen into pTrcHisTOPO vector, PCR was performed using an internal primer pair, ATG TCT ACC GCT GAG CTC GCT G (SEQ ID NO: 5) and GCT TAG TCG AAG AGA CCG AAG C (SEQ ID NO: 6).

Northern Blot Analysis of Clone Pen b 26

About 20 μg total RNA from P. brevicompactum together with RNA molecular weight markers (0.24-9.5 kb RNA Ladder, Invitrogen) were fractioned in 1.3% formaldehyde-agarose gel and transferred to a positively charged nylon membrane in 20×SSC (3 M NaCl, 0.3 M sodium citrate, pH 7.0) with overnight capillary action. RNA was UV-cross-linked to the membrane. DIG Northern blot hybridization was performed according to the manufacturer's instructions (Roche Diagnostics, Basel, Switzerland) with slight modifications (Engler-Blum G. et al. 1993, Anal Biochem 210: 235-244) as follows. Briefly, the membrane was incubated for 1 h in pre-hybridization solution (20% SDS, 0.25 M Na₂HPO₄, 1 mM EDTA and 1% blocking agent). Alkali-labile, DIG-11-dUTP labelled Pen b 26 cDNA probe was prepared by using above PCR. The probe was added to the freshly prepared prehybridization solution at 5 pmol/ml and incubated overnight at 65° C. The probe solution was removed and washed with 2×SSC and 0.4% SDS for 4×5 min intervals at 65° C., followed by a higher stringency wash with 0.5×SSC and 0.1% SDS, 4×5 min intervals at 60° C. The membrane was blocked with 1% blocking solution in wash buffer (3 M NaCl, 0.1 M maleic acid, 0.3% Tween 20, pH 7.5) for 1 h. Anti-DIG-AP conjugate was added into the same freshly made blocking solution at 1/10,000 dilution and incubation continued for another 30 min, followed by 4× for 10 min washes with the wash buffer, and incubated for 2 min in 0.1 M Tris, pH 9.5. Finally, the membrane was incubated briefly with CDP-Star between transparency pages. The excess liquid was removed and exposed to an X-ray film until the bands became clearly visible.

Sequence Analyses of Clone Pen b 26

Amino acid sequence Pen b 26 was analyzed by using various internet softwares, The secondary structure analyses of Pen b 26 protein was carried out using PSIPRED software from University College London, UK web site (McGuffun L. J. et al. 2002, Bioinformatics 16: 404-405). The hydrophobicity (Hoop T. P. and Woods K. R., 1981, Proc Natl Acad Sci USA 78: 3824-3828) and hydrophilicity (Kyte J. and Doolittle R. F. 1982, J Mol Biol 157: 105-132) plots were drawn using the software of Colorado State University, USA web site (http://arbl.cvmbs.colostate.edu/molkit/hydropathy/index.html). The BLAST algorithm was used for homology searches in GenBank, National Centre for Biotechnology Information, USA web site (Altschul S. F. et al. 1997, Nucl Acids Res 25: 3389-3402). MultAlin (from Institut National de la Recherche Agronomique, France), multiple alignment software was used to align the most homologous sequences (Corpet, 1988, Nucl Acids Res 16: 10881-10890). Peptide analyses was performed by EMBOSS Pepstats using European-Bioinformatics Institute web site (Harrison, 2000, inNovations 11: 4-7). Phosphorylati0n sites were predicted using NetPhos 2.0 server of Technical University of Denmark (Blom etal., 1999, J Mol Biol 294: 1351-1362).

Database Submissions

The cDNA and predicted amino acid sequence of Pen b 26 was submitted to GenBank under the accession number AY786077. Data obtained from this study was also submitted to the Allergen Nomenclature Sub-Committee for approval of the new allergen, designated as Pen b 26.

Expression of Pen b 26

The CDNA encoding Pen b 26 was amplified by polymerase chain reaction and cloned into pTrcHisTOPO TA vector (Invitrogen) and transformed into Escherichia coli TOP10 strain (Invitrogen). In this vector, a peptide containing the HisG epitope and a 6× His tag for detection and purification of the recombinant protein was fused into the N-terminus of Pen b 26 (SEQ ID NO: 3 and SEQ ID NO: 4). The N-terminal fused peptide added about 3-4 kDa to the size of Pen b 26, thereby increasing the estimated size of Pen b 26 from 11 kDa to 15 kDa approximately. The cultures of E. coli were grown in LB with 50 ug/ml ampicillin. The expression of the His-tagged Pen b 26 was induced by the addition of IPTG into the culture medium at mid-log phase cultures.

Purification of Pen b 26

Pen b 26 was purified using ProBond purification kit (Invitrogen, cat# K4410-01) designed for nickel-binding properties of histidine-tagged proteins.

Mass Spectral Analysis of Pen b 26

Electrospray

Electrospray ionization Mass spectroscopy was carried out using the affinity chromatography-purified Pen b 26. A Waters/Micromass (Milford, Mass., USA) Q-Tof-1 mass spectrometer with a nanospray (Z-spray) source was used in the positive ion mode. Samples of Pen b 26 were evaporated to dryness under a stream of nitrogen gas. The dried sample was dissolved in 25 ul of 0.2% formic acid in water-methanol (1:1 v/v), and redissolved by sonication at low power. The samples were then centrifuged at 10,000 rpm for 5 minutes before being loaded into Econospray nanospray tips New Objective, Inc. (Woburn, Mass.).

MALDI-TOF

The His-tag affinity chromatography-purified Pen b 26 fusion protein was analysed by MALDI-TOF mass spectrometry. The sample for MALDI-TOF-MS analysis was prepared as reported (Kumarathasan P. et al., Anal Biochem 2005; 346, 85-89). Saturated MALDI matrix solution was prepared by dissolving re-crystallized cinapinic acid (Bruker Daltonics) in 50% acetonitrile in 0.1% TFA (aq). Initially, matrix solution (1 μL) was spotted on an Anchor Chip target plate (600/384F, Bruker Daltonics) followed by the sample (1 μL). The sample-matrix mixture was dried, and the dried sample was washed by 0.1% TFA. The dried sample was analysed by Bruker Autoflex MALDI-TOF mass spectrometer (Bruker Daltonics, Germany). Calibration of the instrument was carried using protein calibration standard mix I (Bruker part #206355).

Results

Isolation of Clone Pen b 26 by Plaque Immunohybridization Assay

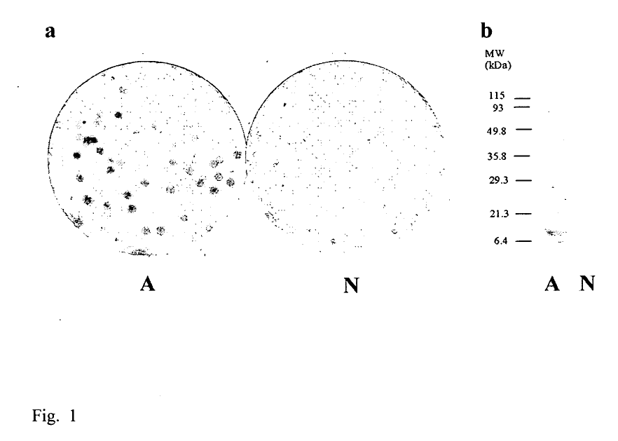

A phage cDNA library of P. brevicompactum was constructed in Uni-ZAP® vector using total RNA isolated from the organism. The phage plaques of the cDNA library of P. brevicompactum were screened using pooled atopic sera from individuals sensitive to P. brevicompactum. Among several allergenic clones, one clone, designated as Pen b 26, was found to be strongly allergenic. This clone was further purified by phage dilution method. The purified clone was found to be only reactive with the atopic sera obtained from individuals sensitive to this mold (FIG. 1a). Pen b 26 phage clone was amplified in E. coli host and a crude extract was made by sonicating the phase particles. The crude extract was analyzed by Western blot as shown in FIG. 1b. The allergenic clone Pen b 26 had an approximate MW weight of 10 kDa.

DNA Sequencing, PCR and Northern Blot Hybridization

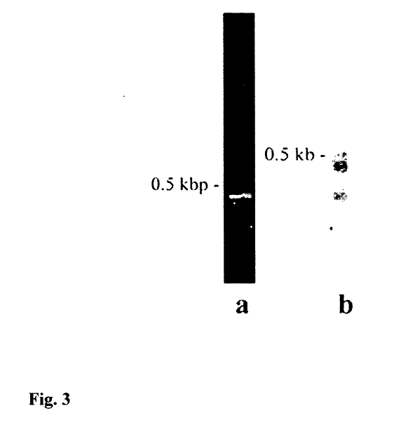

Performing single colony excision protocol, phage clone Pen b 26 was converted to phagemid catalyzed by the helper phage. Phagemid was purified as plasmid in pBluescript vector and further analyzed for DNA sequence and insert size. The DNA sequence of Pen b 26 in pBluescript is shown in FIG. 2. The cDNA insert had a nucleotide sequence of 455 bp. An open reading frame deduced from the cDNA sequence data using the largest predicted sequence encoded a polypeptide of 107 residues with a predicted molecular size of 11 kDa (table 1) which was in close agreement with the Western blot analysis as discussed above (FIG. 1b). Furthermore, a PCR was performed using flanking primers from pBluescript vector (FIG. 3a). A PCR product of about 0.5 kb, including the external primer sequences was obtained, which confirmed the size of the cDNA sequence obtained from the sequence data. The size of the transcript was further confirmed by Northern blot hybridization using the cDNA of clone Pen b 26 as a probe (FIG. 3b). This probe was hybridized strongly with a band of about 0.5 kb. However, there were two more unrelated bands, one is around 0.3 kb and the other one is a little higher than 0.5 kb, cross-reacted with the same probe despite higher stringency washes. Hence, the size of the cDNA fragment was further confirmed.

Protein Sequence Analysis

Pen b 26 protein appears to have strongly acidic and negatively charged residues; 21.5% with an isoelectric point of 3.87 (table 1). This protein was also found to be unusually rich in alanine (20.5%) and glutamic acid (15%), and together they comprised more than one-third of all the residues. Furthermore, the amino acid sequence of Pen b 26 was analyzed using NetPhos 2.0 Server (Blom N. et al. 1999, J Mol Biol 294: 1351-1362) for the prediction of phosphorylated residues. It was found that two potential residues, Ser-97 and Thr-29 had high scores of 0.997 and 0.764, respectively.

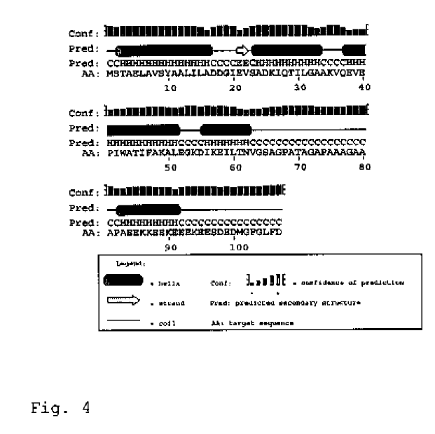

The secondary structure analyses of Pen b 26 protein carried out using PSIPRED software (FIG. 4), which predicted extensive alpha-helical regions from the N-terminus to the middle of the gene intercepted by short coil regions. However, towards the C-terminus larger segment of coil regions appeared. There was little indication of a strand structure closer to the N-terminus.

Furthermore, the hydrophobicity and hydrophilicity plots were analyzed (FIG. 5). Pen b 26 protein had hydrophobic regions closer to its N-terminus, indicating that N-terminus could be buried while strong hydrophilic regions were found towards its C-terminus region. Hydrophilic regions on the protein are exposed on the surface and most likely play a key role in antigenicity and allergenicity.

Amino acid sequence homology analysis of Pen b 26 protein was performed first using BLAST program and about 100 sequences appeared to have homology to Pen b 26. Later, Multalin program was used to align the most prominent examples of the homologous sequences (FIG. 6). Further sequence analysis indicated that Pen b 26 protein had greater than 50% amino acid sequence homology to 60S ribosomal P1 proteins from a variety of species, primarily from fungal sources, including Aspergillus fumigatus, Cladosporium herbarum, Neurospora crassa, Alternana altemata, and Schizosaccharomyces pombe (FIG. 6).

Expression of Pen b 26

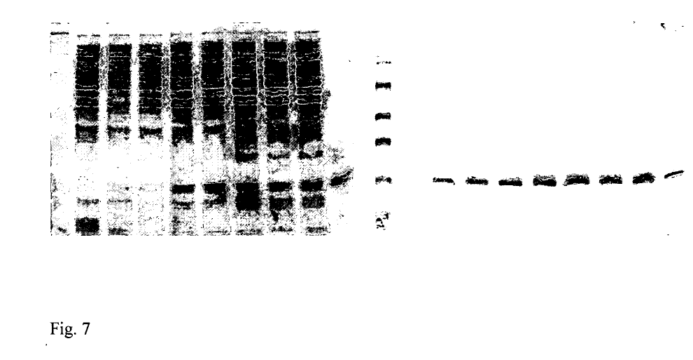

The N-terminal His-tagged Pen b 26 fusion protein was expressed in Escherichia coli successfully. The time-course of the expression was followed and found to be maximum after 3-4 following induction (FIG. 7). The purified preps of Pen b 26 fusion protein were made from the cultures of E. coli after 4 h induction.

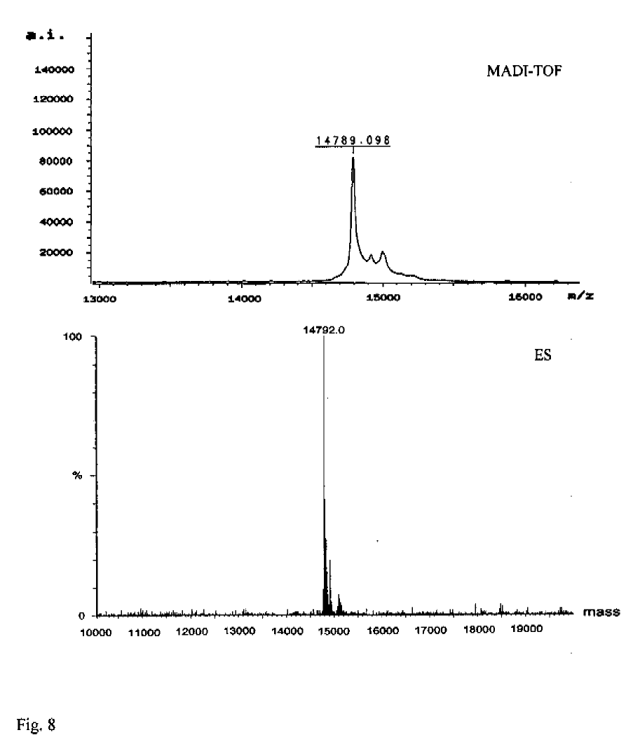

The purified Pen b 26 fusion protein was visualised by SDS-PAGE after commassie brilliant blue stain and western blot hybridization. The fusion protein had about 20-22 kDa apparent size on SDS-PAGE gels while its estimated size was 15 kDa and it always migrated as closely attached but two separate bands in both purified and crude extracts. The discrepancy between the calculated and apparent sizes of Pen b 26 fusion protein was clarified using mass spectroscopy. Mass spectral analyses by both electrospray and MALDI-TOF produced almost identical results and confirmed the calculated size of Pen b 26 fusion protein (FIG. 8). Therefore, the apparent size of Pen b 26 fusion protein as seen on SDS-PAGE may be an artefact.

Confirming that Pen b 26 is a 60S acidic ribosomal P protein

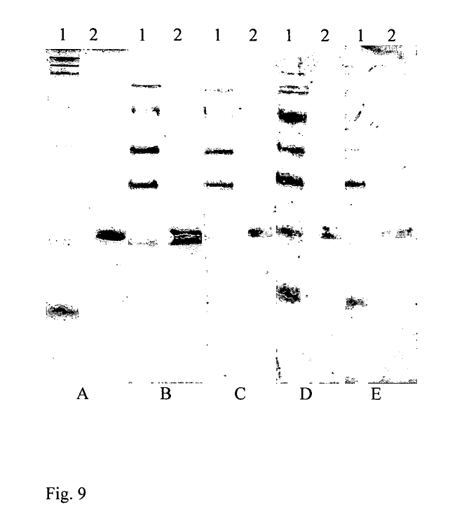

Pen b 26 fusion protein was further analyzed by Western blotting using Human antibody against ribosomal P antigen (Immunovision, Springdale, Ark., USA). The result showed positive reaction indicated that Pen b 26 was indeed a 60S acidic ribosomal P protein (FIG. 9).

Prevalence of allergies against Pen b 26

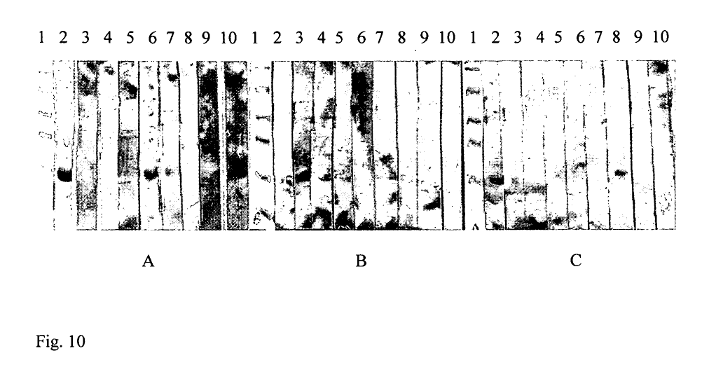

Purified Pen b 26 was used to screen the atopic sera to determine the prevalence allergies against Pen b 26 (FIG. 10). The results obtained from the screening of 27 individual sera indicated that 7 of the Penicillium-sensitive individuals had antibodies against Pen b 26 (25.9%). This data indicated that Pen b 26 was an minor allergen of P. brevicompactum.

While the preferred embodiments of the invention have been described above, it will be recognized and understood that various modifications may be made therein, and the appended claims are intended to cover all such modifications which may fall within the spirit and scope of the invention.

1. An isolated nucleic acid sequence comprising nucleotides 1-455 of SEQ ID NO: 1.

2. An isolated peptide comprising 6 or more consecutive amino acids of SEQ ID NO: 2.

3. The peptide according to claim 2 comprising amino acids 1 to 107 of SEQ ID NO. 2.
